(19) United States
(12) Patent Application Publication
(10) Pub. No.: US 2017/0020930 A1
(43) Pub. Date: Jan. 26, 2017

(54) PREVENTION OF PREGNANCY COMPLICATIONS BY PROBIOTIC ADMINISTRATION

(71) Applicant: THERAPEUTIC SOLUTIONS INTERNATIONAL, INC., Oceanside, CA (US)

(72) Inventors: Thomas Ichim, San Diego, CA (US); Timothy G. Dixon, Oceanside, CA (US)

(21) Appl. No.: 15/215,344

(22) Filed: Jul. 20, 2016

Related U.S. Application Data
(60) Provisional application No. 62/194,990, filed on Jul. 21, 2015.

(51) Int. Cl. A61K 35/744; A61K 35/745; A23L 33/135; A61K 35/747

(52) U.S. Cl. CPC A61K 35/744; A61K 35/747; A61K 35/745; A23L 33/135; A61K 2035/115

(57) ABSTRACT

Methods for the treatment of pregnancy complications through immune modulation are disclosed. Also disclosed are probiotic compositions capable of inducing an anti-inflammatory immune response in a subject. The probiotic composition may include one or more of Streptococcus thermophiles, Lactobacillus reuteri, Bifidobacterium bifidium, Lactobacillus acidophilus, and Lactobacillus casei.

### RELATED APPLICATIONS

The present application claims the benefit of priority to U.S. Provisional Patent Application No. 62/194,990, filed Jul. 21, 2015, the disclosure of which is incorporated by reference herein in its entirety.

### FIELD OF THE DISCLOSURE

The disclosure pertains to the field of correction of immunological abnormalities utilizing probiotics as a means of therapeutic immune modulation. More specifically, the disclosure relates generally to methods for the prevention of immunologically mediated abortions through administration of probiotics. Probiotics are administered based on immunological parameters that are identified in patients at risk of spontaneous miscarriages.

### BACKGROUND

Abortion is classically defined as the termination of pregnancy before the 28th gestational week characterized by expulsion of the fetus or fetal death. Abortion can be classified into early abortion (before the 12th gestational week) and late abortion (after that time point). Spontaneous abortion is the termination of pregnancy due to natural causes, such as some kind of diseases, without artificial interference. Spontaneous abortion includes accidental spontaneous abortion and recurrent spontaneous abortion (RSA), which is two or more consecutive abortions characterized by the termination of fetal development in the same gestational week. It is believed that 2-3% of pregnant women suffer from RSA.

RSAs are believed to be the result of fetal chromosomal abnormalities, endocrine imbalance, anatomical abnormality of reproduction organs, bacterial infection, viral infection, blood group incompatibility between mother and fetus and environmental pollution (Christiansen, O. B., et al. Evidence-based investigations and treatments of recurrent pregnancy loss. Fertil Steril, 2005. 83(4): p. 821-39). About half of RSAs still have no known cause, and are called unexplained RSAs. Improvements in reproductive immunology and the development of immunological assays have led to our current understanding that immunological factors are thought to be the main cause of unexplained RSAs (Teles, A., Zenciussen, A. E., and Schumacher, A. Regulatory T Cells are Baby's Best Friends. Am J Reprod Immunol, 2013. 69(4): p. 331-9). The citations referred to herein are all incorporated by reference in their entireties.

Several representative hypotheses exist about the immunological mechanism of RSA, including for example: production of the blocking antibodies (BA), such as anti-paternal cytotoxic antibodies (APCA), anti-idiotypic antibodies (Ab2) and mixed lymphocyte reaction blocking antibodies (MLR-Bf) which can inhibit the attack to fetus by maternal immunological system, is inhibited due to the increased sharing of human leukocyte antigens (HLA) between the couple (Oksenberg, J. R., et al., Mixed lymphocyte reactivity nonresponsiveness in couples with multiple spontaneous abortions. Fertil Steril, 1983. 39(4): p. 525-9; Lauritsen, J. G., Kristensen, T., and Grunnet, N. Depressed mixed lymphocyte culture reactivity in mothers with recurrent spontaneous abortion. Am J Obstet Gynecol, 1976. 125(1): p. 35-9). This is in fact supported by findings of smaller placental sizes in inbred animal strains compared to outbred animals (McCarthy, J. E. The effects of inbreeding on birth weight and foetal and placental growth in mice. Genet Res, 1968. 11(1): p. 43-9). Another potential cause of RSA is over activity of helper T cell 1 (Th1)-derived cytokines and of natural killer cells (NK) (Kusnierczyk, P. Killer cell immunoglobulin-like receptor gene associations with autoimmune and allergic diseases, recurrent spontaneous abortion, and neoplasms. Front Immunol, 2013. 4: p. 8; Karami, N., et al. Enhancement of peripheral blood CD56(dim) cell and NK cell cytotoxicity in women with recurrent spontaneous abortion or in vitro fertilization failure. J Reprod Immunol, 2012. 95(1-2): p. 87-92; Krigstein, M. and Sacks, G. Prednisolone for repeated implantation failure associated with high natural killer cell levels. J Obstet Gynaecol, 2012. 32(6): p. 518-9). The citations referred to herein are all incorporated by reference in their entireties.

Because the immune recognition mechanism between pregnant woman and fetus has not been fully revealed, the immunological pathogenesis of RSA has not yet been accurately understood. No method of treatment with definite curative effect is available heretofore. Currently, one widely used method for treating immunological RSA is lymphocyte immunotherapy. Immunotherapy of RSA has been applied both in China and other countries since Taylor and Faulk infused a patient with unexplained RSA a suspension of mixed leukocytes derived from her spouse in 1981, which was subsequently confirmed in larger trials (Taylor, C. G., Faulk, W. P., and McIntyre, J. A. Prevention of recurrent spontaneous abortions by leukocyte transfusions. J R Soc Med, 1985. 78(8): p. 623-7, which is incorporated herein by reference in its entirety). For this type of therapy, the immunogen is lymphocytes from the spouse in most cases. The immunotherapy includes isolating lymphocytes from the spouse's venous blood for intracutaneous injection. Alternatively, the condensed leukocytes or whole blood from the spouse can also be intravenously injected. Usually, the immunization is performed every 2 weeks for a total of 2 to 4 times before pregnancy and boosted 1 to 3 times after pregnancy. Twenty years after the application of lymphocyte immunotherapy for treating RSA, a great deal of studies from China and other countries have indicated that the therapeutic effect of this therapy is not definite and the therapy has some serious adverse side effects. In only one of the six studies from 1981 to 1994 on the immunotherapy of RSA was the effectiveness of the immunotherapy demonstrated. There was no statistically significant difference between the therapy group and the control group in the other studies. In addition, the lymphocyte immunotherapy has some serious adverse side effects such as erythrocyte sensitization, thrombocytopenia and intrauterine growth retardation of fetus. Some diseases transmitted by blood, such as AIDS, may be transferred from one individual to another, due to the living cells with intact nuclear materials, and are used in lymphocyte therapy.

Other immune modulatory approaches have been attempted for RSA, which are highlighted in Table 1 below, although to date all either are not reproducibly effective or possess certain drawbacks.

TABLE 1
Clinical Trials of Immune Modulation in RSA

| Intervention | Outcome | Drawback | Ref. |
|---|---|---|---|
| Anti-TNF-alpha | 30 women with RSA treated, | Small study number, cost | Jerzak, M., |
| Antibody, 4 doses | NK activity decreased overall, | and adverse effects of | et al. Ginekol |
| twice weekly before | higher decrease in those that | blanket immune | Pol, 2012. |
| conception | conceived | suppression. | 83(4): p. |
| | | | 260-4. |
| Paternal lymphocyte | 34 women with RSA had a | No efficacy data related | Wilczynski, |
| infusion (PLI) | statistically significant | to conception | J. R. Arch |
| | decrease in interferon gamma | | Immunol |
| | and increase in IL-10 after PLI, | | Ther Exp |
| | but no correlation with | | (Warsz), |
| | pregnancy success | | 2012. 60(2): |
| | | | p. 151-60). |
| Intravenous | live birth rates of 70% (31/44) | No efficacy | Stephenson, |
| Immunoglobulin, | in the IVIG group and 62% | | M. D., et al. |
| preconception to 20 | (28/45) in the control group | | Hum |
| weeks' gestation | (P = 0.503) | | Reprod, |
| | | | 2010. 25(9): |
| | | | p. 2203-9. |
| Low dose aspirin | Of the 147 participants | No efficacy | Clark, P., et |
| | receiving pharmacologic | | al. Blood, |
| | intervention, 32 (22%) | | 2010. |
| | pregnancy losses occurred, | | 115(21): p. |
| | compared with 29 losses (20%) | | 4162-7. |
| | in the 147 subjects receiving | | |
| | intensive surveillance alone | | |
| IVIG therapy in RSA | Clinical pregnancy rate for the | Requires identification of | Moraru, M., |
| women who have | women under IVIG therapy was | women who have high | et al. Am J |
| abnormal expansion of | 92.5% and the live birth rate | NK activity and RSA. | Reprod |
| NK cells | was 82.5%. Significantly lower | IVIG is costly and | Immunol, |
| | pregnancy and live birth | cumbersome to deliver. | 2012. 68(1): |
| | respectively) were observed | | p. 75-84. |
| | for the patients with recurrent | | |
| | pregnancy loss and NK/NKT- | | |
| | like cells expansion without | | |
| | IVIG. | | |
| G-CSF | In the group treated with G- | Small sample size, G- | Scarpeliini, |
| | CSF, 29 out of 35 (82.8%) | CSF is costly and | F. and |
| | women delivered a healthy | potential adverse effects | Sbracia, M. |
| | baby, whereas in the placebo | associated with | Hum |
| | group, this figure was only 16 | neutrophilia | Reprod, |
| | out of 33 (48.5%) (P = 0.0061, | | 2009. |
| | odds ratio = 5.1; 95% | | 24(11): p. |
| | confidence interval 1.5-18.4). | | 2703-8. |
| | Significantly higher beta-hCG | | |
| | levels were found in gestation | | |
| | weeks 5-9 in women treated | | |
| | with G-CSF versus placebo | | |
| | (P < 0.001). | | |
| PLI | Of the 140 newly pregnant | Small sample size, | Nonaka, T., |
| | patients after immunotherapy, | difficult procedure. | et al. Am J |
| | the pregnancy continued | | Reprod |
| | successfully in 110 (78.6%). | | Immunol, |
| | The success rate of pregnancy | | 2007. 58(6): |
| | was 30.0% in 18 non- | | p. 530-6. |
| | immunized patients | | |

The citations referred to in Table 1 are all incorporated by reference in their entireties.

The role of inflammatory processes in pregnancy is not limited to RSA, but spans a wide variety of risk factors. For example, preterm rupture of the membranes (PPROM) and spontaneous (with intact membranes) preterm labor are the most common antecedents of preterm birth. Although there are many causes of preterm birth, intra-amniotic infection and chorioamnionitis appear to be the most significant contributing factors (Menon, R. and Fortunato, S. J. Infection and the role of inflammation in preterm premature rupture of the membranes. Best Pract Res Clin Obstet Gynaecol, 2007. 21(3): p. 467-78). Specifically, spontaneous preterm birth, caused by preterm labor (contractions before 37 weeks' gestation) or PPROM or both account for approximately 80% of preterm deliveries. PPROM is associated with 30-40% of preterm deliveries. It is known that infection and inflammation trigger the activation of inflammatory cytokines, cyclooxygenase (PTGS2) and prostaglandins, and extracellular matrix (ECM) remodeling enzymes (e.g. matrix metalloproteinase-9 (MMP-9)), which are involved in the processes leading to uterine contractions and membrane rupture. Nuclear factor-kappa B (NFKB) is a key pro-inflammatory transcription factor involved in promoting the formation of these pro-inflammatory and pro-labor mediators in human gestational tissues (Lappas, M. NOD1 and NOD2 Regulate Proinflammatory and Prolabor Mediators in Human Fetal Membranes and Myometrium via Nuclear Factor-Kappa B. Biol Reprod, 2013). The citations referred to herein are all incorporated by reference in their entireties.

Cerebral Palsy (CP) occurs as a result of brain injury shortly before or during birth and is characterized by non-progressive hypertonia and/or spasticity. Autopsy samples from patients with CP reveal white-matter injury, germinal matrix hemorrhage with intraventricular extension, and injury to the cortex, basal ganglia, and thalamus (Folkerth, R. D. Neuropathologic substrate of cerebral palsy. J Child Neurol, 2005. 20(12): p. 940-9). Causative factors are believed to be direct ischemia/hypoxia insult (Hankins, G. D. and Speer, M. Defining the pathogenesis and pathophysiology of neonatal encephalopathy and cerebral palsy. Obstet Gynecol, 2003. 102(3): p. 628-36), or in some cases maternal infection (Grether, J. K. and Nelson, K. B. Maternal infection and cerebral palsy in infants of normal birth weight. JAMA, 1997. 278(3): p. 207-11; Neufeld, M. D., et al. Maternal infection and risk of cerebral palsy in term and preterm infants. J Perinatol, 2005. 25(2): p. 108-13), both of which culminate in causing inflammatory cytokine activation in the fetal CNS, demyelination and excitotoxicity. Supporting these mechanisms of CP induction are animal models in which induction of ischemia/hypoxia, glutamate excitotoxicity, or mediators of bacterial/viral pathogens in the perinatal period elicit neuro-behavioral defects similar to CP (Saliba, E. and Marret, S. Cerebral white matter damage in the preterm infant: pathophysiology and risk factors. Semin Neonatol, 2001. 6(2): p. 121-33; Choi, E. K., et al. Animal models of periventricular leukomalacia. Lab Anim Res, 2011. 27(2): p. 77-84; Rousset, C. I., et al. Antenatal bacterial endotoxin sensitizes the immature rat brain to postnatal excitotoxic injury. J Neuropathol Exp Neurol, 2008. 67(10): p. 994-1000). Currently there are no approved treatments for CP. The citations referred to herein are all incorporated by reference in their entireties.

Although some clinical trials have suggested that magnesium sulfate prevents various complications of pregnancy by reducing inflammatory mediators (Clark, E. A. and Varner, M. Impact of preterm PROM and its complications on long-term infant outcomes. Clin Obstet Gynecol, 2011. 54(2): p. 358-69; Dowling, O., et al. Magnesium sulfate reduces bacterial LPS-induced inflammation at the maternal-fetal interface. Placenta, 2012. 33(5): p. 392-8; Rochelson, B., et al. Magnesium sulfate suppresses inflammatory responses by human umbilical vein endothelial cells (HuVECs) through the NFkappa8 pathway. J Reprod Immunol, 2007. 73(2): p. 101-7; Burd, I., et al. Magnesium sulfate reduces inflammation-associated brain injury in fetal mice. Am J Obstet Gynecol, 2010. 202(3): p. 292 el-9; Sugimoto, J., et al. Magnesium decreases inflammatory cytokine production: a novel innate immunomodulatory mechanism. J Immunol, 2012. 188(12): p. 6338-46), adverse effects have been associated with its use, and overall efficacy has not been proven (Smith, J. M., et al. An integrative review of the side effects related to the use of magnesium sulfate for pre-eclampsia and eclampsia management. BMC Pregnancy Childbirth, 2013. 13: p. 34). The citations referred to herein are all incorporated by reference in their entireties.

### SUMMARY

The present disclosure is directed to methods for the treatment of pregnancy complications through immune modulation and probiotic compositions capable of inducing an anti-inflammatory immune response in a subject.

In some embodiments is provided a method for reducing a pregnancy complication or a risk of a pregnancy complication in a subject including administering to the subject a probiotic composition. In some embodiments, the probiotic composition includes Lactobacillus, Bifidobacterium, Escherichia, Saccharomyces, Streptococcus, Bacillus, or combinations thereof In some embodiments, the probiotic composition includes one or more bacteria selected from the group including Streptococcus thermophiles, Lactobacillus reuteri, Bifidobacterium bifidium, Lactobacillus acidophilus, and Lactobacillus casei. In some embodiments, the probiotic composition consists essentially of Streptococcus thermophiles, Lactobacillus reuteri, Bifidobacterium bifidium, Lactobacillus acidophilus, and Lactobacillus casei.

In some embodiments, the pregnancy complication or risk of a pregnancy complication is selected from the group including recurrent spontaneous abortions (RSA) or risk of RSA, preterm birth, low birth weight, preeclampsia, hemolysis elevated liver enzymes low platelets (HELP), premature rupture of the membrane, antepartum hemorrhage, placental abruption, chorioamnionitis, intrauterine growth restriction, placenta previa, sequelae of intraamniotic infection, and cerebral palsy.

In some embodiments, RSA or the risk of RSA includes one or more symptoms selected from the group including having one or more miscarriages in the first trimester of pregnancy, having a higher natural killer cell activity compared to an age-matched group of women with one or more successful pregnancies, having a deficient T regulatory cell activity compared to an age-matched group of women with one or more successful pregnancies, having a higher number of circulating natural killer cells as compared to a group of age-matched women with one or more successful pregnancies, and having a lower number of circulating T regulatory cells as compared to a group of age-matched women with one or more successful pregnancies.

In some embodiments, the natural killer cell activity includes an ability to induce death in vitro in a cell type susceptible to natural killer cell mediated killing. In some embodiments, the natural killer cells express a marker selected from the group including CD16, CD56, perforin, and CD94.

In some embodiments, T regulatory cell activity is quantified by an ability to inhibit a mixed lymphocyte reaction, an ability to inhibit proliferation of a lymphocyte after stimulation or an ability to inhibit cytokine production of a lymphocyte after stimulation. In some embodiments, the cytokine is selected from the group including Interferon gamma, TNF-alpha, IL-12, IL-15, IL-17, IL-2, and IL-21. In some embodiments, the T regulatory cells possess the ability to stimulate production of an anti-inflammatory cytokine selected from the group including IL-4, IL-10, IL-13, IL-20, and TGF-β. In some embodiments, the T regulatory cells express a marker selected from the group including FoxP3, TGF-beta, LAG, and CD73.

In some embodiments, the pregnancy complication or risk of pregnancy complication is preterm birth. In some embodiments, preterm birth is birth before 37 weeks of gestation. In some embodiments, the risk of preterm birth includes an increased vaginal or systemic concentration of one or more compound selected from the group including sialidase, prolidase, glycosyltransferase types I, II and IV, monocyte chemotactic protein-1, matrix metalloproteases I, VIII and IX, IP-10, IL-6, IL-1 beta, TNF-alpha, fetal fibronectin; thrombin-antithrombin complex, and salivary estriol, as compared to a group of age-matched women having one or more successful pregnancies.

In some embodiments, the risk of preterm birth includes a decreased vaginal or systemic concentration of one or more compound selected from the group including maternal serum placental leucine amniopeptidase (P-LAP), IL-10, insulin-like growth factor-binding protein-1 (IGBP-1), Pregnancy associated plasma protein-A (PAPPA), and Corticotropin-releasing hormone (CRH), as compared to a group of age-matched women having one or more successful pregnancies.

In some embodiments is provided a probiotic composition. In some embodiments, the probiotic composition includes Lactobacillus, Bifidobacterium, Escherichia, Saccharomyces, Streptococcus, Bacillus, or combinations thereof.

In some embodiments, the probiotic composition includes a lactic acid bacterium. In some embodiments, the lactic acid bacterium is selected from the group including Lactobacillus, Leuconostoc, Pediococcus, Lactococcus, Aerococcus, Carnobactehum, Enterococcus, Oenococcus, Teragenococcus, Vagococcus, and Weisella.

In some embodiments, the probiotic composition includes one or more bacteria selected from the group including Streptococcus thermophiles, Lactobacillus reuteri, Bifidobacterium bifidium, Lactobacillus acidophilus, and Lactobacillus casei.

In some embodiments, the probiotic composition consists essentially of Streptococcus thermophiles, Lactobacillus reuteri, Bifidobacterium bifidium, Lactobacillus acidophilus, and Lactobacillus casei.

In some embodiments, the probiotic composition is 10⁴, 10⁵, 10⁶, 10⁷, 10⁸, 10⁹, 10¹⁰, 10¹¹, 10¹², 10¹³, 10¹⁴, or 10¹⁵ colony forming units (CFU) bacteria/gram, or a value within a range defined by any two of the aforementioned values.

In some embodiments, the probiotic combination is administered orally. In some embodiments, the probiotic combination is administered together with a food supplement. In some embodiments, the dosage and frequency of administration of the probiotic composition is based on an immune response of the subject. In some embodiments, said immune response is measured using immune parameters selected from the group including T cell proliferative response to a mitogen, T cell cytokine production in response to a mitogen, NK cytotoxicity, level of complement fixing antibodies, B cell production of complement fixing antibodies, and production of inflammatory cytokines.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments, and are not intended to be limiting in scope.

FIG. 1 is a graphical representation of the inhibition of immune mediated resorption by probiotic mix in mice, based on the data provided in Table 2.

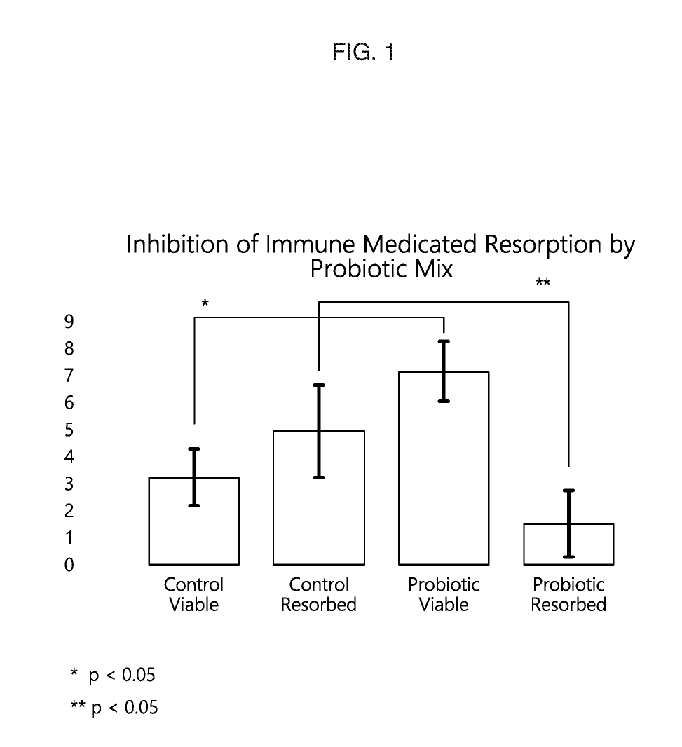

### DETAILED DESCRIPTION

The present disclosure teaches the use of various probiotics and compositions to induce immune modulation systemically, or in some cases locally in a mammal susceptible to pregnancy complications. Probiotics useful for the practice of these disclosed methods cause immune modulation locally and/or systemically depending on the specific embodiment. Particularly, probiotics are chosen based on ability to counteract immunological abnormalities associated with RSA. Specific abnormalities include: elevated natural killer cell numbers (Kwak, J. Y., et al. Up-regulated expression of C056+, C056+/C016+, and C019+ cells in peripheral blood lymphocytes in pregnant women with recurrent pregnancy losses. Am J Reprod Immunol, 1995. 34(2): p. 93-9), reduced number of T regulatory cells (Teles, A., Zenciussen, A. C., and Schumacher, A. Regulatory T Cells are Baby's Best Friends. Am J Reprod Immunol, 2013. 69(4): p. 331-9; Lee, S. K., et al., Th17 and regulatory T cells in women with recurrent pregnancy loss. Am J Reprod Immunol, 2012. 67(4): p. 311-8; Yin, Y., et al. Adoptive transfer of C04+C025+ regulatory T cells for prevention and treatment of spontaneous abortion. Eur J Obstet Gynecol Reprod Biol, 2012. 161(2): p. 177-81) and increased Th1/Th17 cytokines (Matthiesen, L., Kalkunte, S., and Sharma, S. Multiple pregnancy failures: an immunological paradigm. Am J Reprod Immunol, 2012. 67(4): p. 334-40). The citations referred to herein are all incorporated by reference in their entireties.

In one embodiment, inflammatory and immunological abnormalities are identified in order to categorize risk of pregnancy complications, said pregnancy complications are defined as medical incidences that threaten the health of the mother or the offspring, and include RESA, preterm birth, pre-eclampsia including hemolysis elevated liver enzymes low platelets (HELP), premature rupture of the membrane, Antepartum hemorrhage including placental abruption, chorioamnionitis, intrauterine growth restriction, placenta previa, sequelae of intraamniotic infection. In one particular embodiment, levels of circulating factors are assessed in maternal plasma, based on abnormally high levels, interventions are chosen for treatment. In one particular embodiment, the methodology of Ruiz et al. (Ruiz, R. J., et al., Second trimester maternal plasma levels of cytokines /L-1Ra, 11-6 and /L-10 and preterm birth. J Perinatol, 2012. 32(7): p. 483-90, which is incorporated herein by reference in its entirety), is utilized for assessment of circulating IL-6. Specifically, plasma is analyzed in the second trimester of pregnancy and concentrations correlated with a baseline associated with non-complicated pregnancy. Within the context of the present disclosure, other markers of inflammation may be utilized such as C reactive protein (Sorokin, Y., et al. Maternal serum interleukin-6, l C-reactive protein, and matrix metalloproteinase-9 concentrations as risk factors for preterm birth<32 weeks and adverse neonatal outcomes. Am J Perinatol, 2010. 27(8): p. 631-40, which is incorporated herein by reference in its entirety). In females who have higher concentration of inflammatory proteins as compared to baseline values from non-complicated pregnancies, an agent is administered to reduce inflammation. In one embodiment, a probiotic is utilized, in other embodiments, vitamins, cytokines, small molecules, antibodies, aptamers, or other means of suppressing inflammation are utilized. Various probiotics possess immune modulatory properties. For example, the oral administration of a combination of five probiotic strains in equal ratio reduced clinical symptoms and suppressed levels of autoreactive T cells in a mouse model of autoimmunity, called experimental autoimmune myasthenia gravis (EAMG). The five probiotic strains were Streptococcus thermophilus (ST), Lactobacillus reuteri (LR), Bifidobacterium bifidium (BB), Lactobacillus acidophilus (LA) and, Lactobacillus casei (LC) The suppression of auto reactivity at the T cell level was associated with the generation of regulatory dendritic cells (rDCs) that express increased levels of IL-10, TGF-β, and arginase 1. Furthermore, DCs isolated from probiotics-fed group effectively converted CD4+T cells into CD4+Foxp3+ regulatory T cells compared with control DCs, thus suggesting an “infectious tolerance” quality of immune modulation induced by oral probiotic administration. The present disclosure teaches that the use of such probiotic approaches to induce immune modulation may be applied to conditions of pregnancy complications to evoke a systemic immune modulation useful for promoting cellular and cytokine pathways that are protective of pregnancy, and to inhibit cellular and cytokine pathways that are pathological to pregnancy.

Accordingly, provided herein are methods of reducing pregnancy complications or the risk of pregnancy complications including, for example, administering an immune modulatory agent or treatment procedure capable of immune modulating a mammal at risk. In some embodiments, the pregnancy complication or risk of pregnancy complication is selected from the group consisting of women at risk for recurrent spontaneous abortions (RSA), preterm birth, low birth weight, preeclampsia including hemolysis elevated liver enzymes low platelets (HELP), premature rupture of the membrane, antepartum hemorrhage including placental abruption, chorioamnionitis, intrauterine growth restriction, placenta previa, sequalae of intraamniotic infection, and cerebral palsy. In some embodiments, the risk of recurrent spontaneous abortion is defined as having one or more miscarriages in the first trimester of pregnancy.

In some embodiments, the risk of pregnancy complications is defined by a deviation in the vaginal microbiome composition, which is significantly different from the normal decrease in community richness and Shannon diversity index of the vaginal microbiome of pregnant women as compared to a group of age-matched non-pregnant women. In some embodiments, the normal decrease in community richness and Shannon diversity index of the vaginal microbiome is defined as having an overall predominance of bacteria, from most to least, of the order Lactobacillales (and family Lactobacillaceae), Clostridiales, Bacteroidales, and Actinomycetales.

In some embodiment, the risk of recurrent spontaneous abortion is defined as having a higher natural killer cell activity compared to an age-matched group of women with one or more successful pregnancies. In some embodiments, the natural killer cell activity is defined as ability to induce death in vitro in a cell type susceptible to natural killer cell mediated killing.

In some embodiments, the risk of recurrent spontaneous abortion is defined as having a higher number of circulating natural killer cells as compared to a group of age-matched women with one or more successful pregnancies. In some embodiments, the natural killer cells express a marker selected from the group consisting of CD16, CD56, perforin, and CD94.

In some embodiments, the risk of recurrent spontaneous abortion is defined as having a deficient T regulatory cell activity compared to an age-matched group of women with one or more successful pregnancies. In some embodiments, the T regulatory cell activity is quantified by ability to inhibit a mixed lymphocyte reaction. In some embodiments, the T regulatory cell activity is quantified by ability to inhibit proliferation of a lymphocyte after stimulation. In some embodiments, the T regulatory cell activity is quantified by ability to inhibit cytokine production of a lymphocyte after stimulation. In some embodiments, the cytokine is selected from the group consisting of Interferon gamma, TNF-α, IL-12, IL-15, IL-17, IL-2 and IL-21. In some embodiments, the T regulatory cell possess an ability to stimulate production of an anti-inflammatory cytokine selected from the group consisting of IL-4, IL-10, IL-13, IL-20, and TGF-β.

In some embodiments, the risk of recurrent spontaneous abortion is defined as having a lower number of circulating T regulatory cells as compared to a group of age-matched women with one or more successful pregnancies. In some embodiments, the T regulatory cells express a marker selected from the group consisting of FoxP3, TGF-beta, LAG, and CD73.

In some embodiments, preterm birth is defined as birth before 37 weeks of gestation. In some embodiments, the risk of preterm birth is defined as possessing an increased vaginal or systemic concentrations of sialidase, prolidase, glycosyltransferase types I, II and IV, monocyte chemotactic protein-1, matrix metalloproteases I, VIII and IX, IP-10, IL-6, IL-1 beta, TNF-α, fetal fibronectin, thrombin-antithrombin complex, salivary estriol or combinations thereof as compared to a group of age-matched women having one or more successful pregnancies. In some embodiments, the risk of preterm birth is defined as possessing decreased vaginal or systemic concentrations of maternal serum placental leucine amniopeptidase (P-LAP), IL-10, insulin-like growth factor-binding protein-1 (IGBP-1), Pregnancy associated plasma protein-A (PAPPA), corticotropin-releasing hormone (CRH) as compared to a group of age-matched women having one or more successful pregnancies. In some embodiments, the agents associated with reducing a risk factor of pregnancy are selected from the group consisting of anti-inflammatory agents, immune modulatory agents, and probiotic agents.

In some embodiments is provided an immune modulatory agent. In some embodiments, the immune modulatory agent is a probiotic composition. In some embodiments, the probiotic composition is a combination of probiotic cultures. In some embodiments, the probiotic composition includes lactic acid bacteria. In some embodiments, the lactic acid bacteria is selected from a group consisting of Lactobacillus, Leuconostoc, Pediococcus, Lactococcus, Aerococcus, Carnobactehum, Enterococcus, Oenococcus, Teragenococcus, Vagococcus, and Weisella.

In some embodiments, the probiotic bacteria is selected from the group consisting of Streptococcus thermophiles, Lactobacillus reuteri, Bifidobacterium bifidium, Lactobacillus acidophilus, Lactobacillus casei, or combinations thereof.

In some embodiments, the probiotic is administered together with a food supplement. In some embodiments, the probiotic is administered through a route selected from the group consisting of orally, intrarectally, intranasally, subcutaneously, intravenously, intramuscularly, and intravaginally. In some embodiments, the dose and frequency of administered probiotic is based on an immune response of the recipient. In some embodiments, the immune response is measured using a variety of immune parameters selected from the group consisting of T cell proliferative response to a mitogen, T cell cytokine production in response to a mitogen, NK cytotoxicity, level of complement fixing antibodies, B cell production of complement fixing antibodies, and production of inflammatory cytokines.

In some embodiments, inflammatory cytokines are selected from the group consisting of BLC, Eotaxin-1, Eotaxin-2, G-CSF, GM-CSF, 1-309, ICAM-1, IFN-gamma, IL-1 alpha, IL-1 beta, IL-1 ra, IL- 2, IL-4, IL-S, IL-6, IL-6 sR, IL-7, IL-8, IL-10, IL-11, IL-12 p40, IL-12 p70, IL-13, IL-15, IL-16, IL-17, MCP-1, MCSF, MIG, MIP-1 alpha, MIP-1 beta, MIP-1 delta, PDGF-BB, RANTES, TIMP-1, TIMP-2, TNF alpha, TNF beta, sTNFRI, sTNFRIIAR, BDNF, bFGF, BMP-4, BMP-S, BMP-7, b-NGF, EGF, EGFR, EG-VEGF, FGF-4, FGF-7, GDF-1S, GDNF, growth hormone, HB-EGF, HGF, IGFBP-1, IGFBP-2, IGFBP-3, IGFBP-4, IGFBP-6, IGF-1, insulin, M-CSF R, NGF R, NT-3, NT-4, osteoprotegerin, PDGF-AA, PIGF, SCF, SCF R, TGF-alpha, TGF beta 1, TGF beta 3, VEGF, VEGFR2, VEGFR3, VEGF-D 6Ckine, Axl, BTC, CCL28, CTACK, CXCL16, ENA-78, Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-2, MCP-3, MCP-4, MDC, MIF, MIP-3 alpha, MIP-3 beta, MPIF-1, MSP-alpha, NAP-2, osteopontin, PARC, PF4, SDF-1 alpha, TARC, TECK, TSLP 4-1BB, ALCAM, B7-1, BCMA, CD14, CD30, CD40 Ligand, CEACAM-1, DR6, Dtk, endoglin, ErbB3, E-Selectin, Fas, Flt-3L, GITR, HVEM, ICAM-3, IL-1 R4, IL-1 RI, IL-10 Rbeta, IL-17R, IL-2Rgamma, IL-21R, L1MPII, Lipocalin-2, L-Selectin, LYVE-1, MICA, MICB, NRG1-betal, PDGF Rbeta, PECAM-1, RAGE, TIM-1, TRAIL R3, trappin-2, uPAR, VCAM-1, XEDARActivin A, AgRP, angiogenin, Angiopoietin 1, angiostatin, catheprin S, CD40, Cripto-1, DAN, DKK-1, E-cadherin, EpCAM, Fos ligand, Fcg RIIB/C, follistatin, galectin-7, ICAM-2, IL-13 R1, IL-13R2, IL-17B, IL-2 Ra, IL-2 Rb, IL-23, LAP, NrCAM, PAI-1, PDGF-AB, Resistin, SDF-1 beta, sgp130, ShhN, Siglec-S, ST2, TGF beta 2, Tie-2, TPO, TRAIL R4, TREM-1, VEGF-C, VEGFR1Adiponectin, adipsin, AFP, ANGPTL4, B2M, BCAM, CA12S, CA1S-3, CEA, CRP, ErbB2, follistatin, FSH, GRO alpha, beta HCG, IGF-1 sR, IL-1 sRII, IL-3, IL-18 Rb, IL-21, leptin, MMP-1, MMP-2, MMP-3, MMP-8, MMP-9, MMP-1O, MMP-13, NCAM-1, nidogen-1, NSE, OSM, procalcitonin, prolactin, PSA, Siglec-9, TACE, thyroglobulin, TIMP-4, TSH2B4, ADAM-9, angiopoietin 2, APRIL, BMP-2, BMP-9, CSa, cathepsin L, CD200, CD97, chemerin, DcR3, FABP2, FAP, FGF-19, Galectin-3, HGF R, IFN-gammalpha/beta R2, IGF-2, IGF-2 R, IL-1R6, IL-24, IL-33, kallikrein 14, legumain, LOX-1, MBL, neprilysin, Notch-1, NOV, osteoactivin, PD-1, PGRP-S, Serpin A4, sFRP-3, thrombomodulin, TLR2, TRAIL R1, transferrin, WIF-1ACE-2, albumin, AMICA, angiopoietin 4, BAFF, CA19-9, CD163, clusterin, CRTAM, CXCL14, cystatin C, decorin, Dkk-3, DLL1, fetuin A, aFGF, FOLR1, furin, GASP-1, GASP-2, GCSF R, HAI-2, IL-17B R, IL-27, LAG-3, LDL R, pepsinogen I, RBP4, SOST, Syndecan-1, TACI, TFPI, TSP-1, TRAIL R2, TRANCE, troponin I, uPA, VE-Cadherin, WISP-1, and RANK.

In some embodiments is provided a probiotic composition useful for reducing a pregnancy complication or a risk of a pregnancy complication. In some embodiments, the composition is capable of inducing an anti-inflammatory immune response in a host. In some embodiments, the anti-inflammatory immune response is represented by an increase in T regulatory cells in a host.

In some embodiments, the probiotics are chosen from a family of probiotic species including Lactobacillus, Bifidobacterium, Escherichia, Saccharomyces, Streptococcus, and Bacillus, or combinations thereof. In some embodiments, Lactobacillus is selected from the group consisting of Lactobacillus acidophilus NCFM, Lactobacillus casei, Lactobacillus casei Shirota, Lactobacillus casei immunitass, Lactobacillus johnsonii, Lactococcus lactis, Lactobacillus plantarum, Lactobacillus reuteri, Lactobacillus rhamnosus, Lactobacillus salivarius, and Lactobacillus helvetirus. In some embodiments, the probiotic is a lactic acid bacterium is selected from the group consisting of Lactobacillus, Leuconostoc, Pediococcus, Lactococcus, Streptococcus, Aerococcus, Carnobactehum, Enterococcus, Oenococcus, Teragenococcus, Vagococcus, and Weisella.

In some embodiments, the composition is administered intravaginally. In some embodiments, the composition is administered orally.

In some embodiments is provided a method for preventing complications of pregnancy including the steps of identifying a mammal at risk for pregnancy complications, administering agents capable of reducing risk factors associated with pregnancy complications, and monitoring the effects of said administered agents so as to “tailor” dosage, type, and frequency of said administered agents.

In some embodiments, the risk factors associated with pregnancy complications is defined as possessing an increased vaginal or systemic concentration of sialidase, prolidase, glycosyltransferase types I, II, and IV, monocyte chemotactic protein-1, matrix metalloproteases I, VIII, and IX, IP-10, IL-6, IL-1 beta, TNF-alpha, fetal fibronectin, and thrombin-antithrombin complex, and salivary extriol, as compared to a group of age-matched women having one or more successful pregnancies.

In some embodiments, the risk of preterm birth is defined as possessing a decreased vaginal or systemic concentration of maternal serum placental leucine aminopeptidase (P-LAP), IL-10, insulin-like growth factor-binding protein-1 (IGBP-1), pregnancy associated plasma protein-A (PAPP-A), and corticotropin-releasing hormone (CRH) as compared to a group of age-matched women having one or more successful pregnancies.

In some embodiments, the agents associated with reducing risk factor of pregnancy are selected from a group consisting of anti-inflammatory agents, immune modulatory agents, and probiotic agents.

Numerous studies have demonstrated that RSA is associated with increased production of Th1 cytokines such as interferon gamma and reduced production of IL-10 (Gharesi-Fard, B., Zolghadri, J., and Kamali-Sarvestani, E. Effect of leukocyte therapy on tumor necrosis factor-alpha and interferon-gamma production in patients with recurrent spontaneous abortion. Am J Reprod Immunol, 2008. 59(3): p. 242-50; Graphou, O., et al. Effect of intravenous immunoglobulin treatment on the Th1/Th2 balance in women with recurrent spontaneous abortions. Am J Reprod Immunol, 2003. 49(1): p. 21-9). Furthermore, treatments that have demonstrated some signal of efficacy in RSA such as IVIG (Yamada, H., et al. Intravenous immunoglobulin treatment in women with recurrent abortions: increased cytokine levels and reduced Th1/Th2 lymphocyte ratio in peripheral blood. Am J Reprod Immunol, 2003. 49(2): p. 84-9), G-CSF (Hartung, T., et al. Effect of granulocyte colony-stimulating factor treatment on ex vivo blood cytokine response in human volunteers. Blood, 1995.85(9): p. 2482-9), and PLT (Liang, P., et al., Comprehensive analysis of peripheral blood lymphocytes in 76 women with recurrent miscarriage before and after lymphocyte immunotherapy. Am J Reprod Immunol, 2012.68(2): p. 164-74), all have been shown to induce a Th1 to Th2 shift. Within the context of the present disclosure, use of probiotic mixtures for inducing immune modulation for protecting the fetal allograft are envisioned. In one specific embodiment, the combination of 5 probiotic strains mentioned above is administered into a mammal suffering from RSA at a concentration sufficient to evoke a therapeutic response. Such concentrations may be determined by monitoring NK activity, assessing inflammatory cytokine production by peripheral blood mononuclear cells after stimulation with a mitogen or mitogenic antibody, or by assessment of T regulatory (Treg) cell numbers or activity. In one embodiment RSA patients are administered the probiotic cocktail at approximately 10 billion bacteria per day. The citations referred to herein are all incorporated by reference in their entireties.

In one embodiment, specific probiotic bacteria are administered individually or in combination with other bacteria. The lactic acid bacterium and/or Bifidobacterium that are used in accordance with the disclosure may include from 10⁶ to 10¹² colony forming units (CFU) of bacteria per gram of support material, and more particularly from 10⁸ to 10¹² CFU of bacteria/gram of support material, preferably 10⁹ to 10¹² CFU/gram of support material for the lyophilized form.

Suitably the lactic acid bacterium and/or Bifidobacterium used in accordance with the methods of the present disclosure may be administered at a dosage of from about 10⁶ to about 10¹² CFU of microorganism/dose, preferably about 10⁸ to about 10¹² CFU of microorganism/dose. By the term “per dose” it is meant that this amount of microorganism is provided to a subject either per day or per intake, preferably per day. For example, if the microorganism is to be administered in a food product (for example in a yoghurt), then the food product will preferably contain from about 10⁸ to 10¹² CFU of the microorganism. Alternatively, however, this amount of microorganism may be split into multiple administrations each consisting of a smaller amount of microbial loading, so long as the overall amount of microorganism received by the subject in any specific time (for instance each 24 hour period) is from about 10⁶ to about 10¹² CFU of microorganism, preferably 10⁸ to about 10¹² CFU of microorganism.

In accordance with the present disclosure an effective amount of at least one strain of a microorganism may be at least 10⁶ CFU of microorganism/dose, preferably from about 10⁶ to about 10¹² CFU of microorganism/dose, preferably about 10⁸ to about 10¹² CFU of microorganism/dose. In one embodiment, preferably the lactic acid bacterium and/or Bifidobacterium used in accordance with the present methods and compositions of the present disclosure (such as a strain of Lactobacillus spp.; for example a strain of Lactobacillus acidophilus, Lactobacillus salivarius and/or Lactobacillus plantarum and/or a strain of Bifidobacterium spp., such as a strain of Lactobacillus acidophilus or Lactobacillus salivarius, for example Lactobacillus acidophilus strain such as NCFM or Lactobacillus salivarius strain 33) such as a strain of Bifidobacterium animalis subsp. lactis, for example Bifidobacterium animalis subsp. lactis strain 420 (B420)) may be administered at a dosage of from about 10⁶ to about 10¹² CFU of microorganism/day, preferably about 10⁸ to about 10¹² CFU of microorganism/day. Hence, the effective amount in this embodiment may be from about 10⁶ to about 10¹² CFU of microorganism/day, preferably about 10⁸ to about 10¹² CFU of microorganism/day.

Disclosed are methods, diagnostic means, and compositions of matter and therapeutics for the diagnosis and treatment of pregnancy complications through immune modulation of a mammal in need. In one embodiment a method of the present disclosure provides assays for establishing risk of pregnancy complications, said assays being utilized to provide therapeutic interventions to ensure successful pregnancies. Pregnancy complications include RSA, preterm birth, pre-eclampsia including HELP, premature rupture of the membrane, Antepartum hemorrhage including placental abruption, chorioamnionitis, intrauterine growth restriction, placenta previa, sequelae of intraamniotic infection.

The probiotic mixtures may be used in accordance with the methods and compositions of the present disclosure in the form of solid or liquid preparations or alternatives thereof Examples of solid preparations include, but are not limited to tablets, capsules, dusts, granules and powders which may be wettable, spray-dried or freeze-dried. Examples of liquid preparations include, but are not limited to, aqueous, organic or aqueous-organic solutions, suspensions and emulsions. Suitable examples of forms include one or more of: tablets, pills, capsules, ovules, solutions or suspensions, which may contain flavoring or coloring agents, for immediate-, delayed-, modified-, sustained-, pulsed- or controlled release applications. By way of example, if a composition of the present disclosure is used in a tablet form—such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycolate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included. Furthermore, examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethylcellulose, polyvinylpyrrolidone, and the like. Preferred excipients for the forms include lactose, starch, a cellulose, milk sugar or high molecular weight polyethylene glycols.

In one embodiment, probiotic bacteria are administered in the form of a nutraceutical. Nutraceuticals, whether in the form of a liquid extract or dry composition, are edible and may be eaten directly by humans or mammals. Said nutraceuticals are preferably provided to humans in the form of additives or nutritional supplements e.g., in the form of tablets of the kind sold in health food stores, or as ingredients in edible solids, more preferably processed food products such as cereals, breads, tofu, cookies, ice cream, cakes, potato chips, pretzels, cheese, etc., and in drinkable liquids e.g., beverages such as milk, soda, sports drinks, and fruit juices. Thus, in one embodiment a method is provided for enhancing the nutritional value of a food or beverage by intermixing the food or beverage with a nutraceutical in an amount that is effective to enhance the nutritional and probiotic or immune modulatory value of the food or beverage. In one embodiment, a flavoring agent is added. Preferred flavoring agents include sweeteners such as sugar, corn syrup, fructose, dextrose, maltodextrose, cyclamates, saccharin, phenyl-alanine, xylitol, sorbitol, maltitol, and herbal sweeteners such as Stevia. Examples of foods into which probiotics useful for the practice of the methods disclosed herein can be incorporated into include soft drinks, a fruit juice or a beverage including whey protein, health teas, cocoa drinks, milk drinks and lactic acid bacteria drinks.

The nutraceuticals described herein are intended for human consumption and thus the processes for obtaining them are preferably conducted in accordance with Good Manufacturing Practices (GMP) and any applicable government regulations governing such processes. Especially preferred processes utilize only naturally derived solvents. In contrast to nutraceuticals, the so-called “medical foods” are not meant to be used by the general public and are not available in stores or supermarkets. Medical foods are not those foods included within a healthy diet to decrease the risk of disease, such as reduced-fat foods or low-sodium foods, nor are they weight loss products. A physician prescribes a medical food when a patient has special nutrient needs in order to manage a disease or health condition, and the patient is under the physician's ongoing care. The label must clearly state that the product is intended to be used to manage a specific medical disorder or condition. An example of a medical food is nutritionally diverse medical food designed to provide targeted nutritional support for patients with chronic inflammatory conditions. Active compounds of this product are for instance one or more of the compounds described herein. The present disclosure thus relates to and teaches methods for use of an immuno-modulating properties of probiotics as related to prevention and/treatment of pregnancy complications. Thus said probiotics can be used in the preparation of a medicament, a vaginal suppository, medical food or nutraceutical to induce immune tolerance or immune modulation.

The preferred embodiments described herein are intended to illustrate the principles of the disclosure, but not to limit its scope. Other embodiments and variations to the preferred embodiments will be apparent to those skilled in the art and may be made without departing from the spirit and scope of the invention as defined in the claims.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

These Examples demonstrate the effectiveness of probiotic combinations as disclosed herein for reducing the risk of pregnancy complications.

### Example 1

### Probiotic Composition Reduces Resorbed Fetus in Mice

The established CBA×DBA mouse model of immunologically mediated spontaneous abortion, was utilized to assess effects of probiotic administration on resorption at day 15.

Wild type 8-10 week old virgin CBA/J female mice and 8-14 week old DBA/2J male mice were paired and vaginal plug was assessed two times a day. Day of formation of the vaginal plug was designated as day zero of pregnancy. Ten pregnant female mice were orally administered a probiotic mixture of L. acidophilus, L. casei, Lactobacillus reuteri, Bifidobacterium bifidium, and Streptococcus thermophiles at a dosage of 5×10⁸ CFU/day. Another 10 mice were used as controls and treated with saline.

Administration was performed daily from day zero of pregnancy until day 15 of pregnancy, when animals were sacrificed and uterine horns were examined for presence of resorbed offspring. Resorption was expressed as number of resorptions/total number of formed fetuses and resorptions.

As seen in Table 2, a significant decrease of resorbed offspring was observed in mothers administered the probiotic mixture. The control treated group on average had 3.2 viable offspring (standard deviation of 1.03), and 4.9 resorbed fetuses (standard deviation of 1.73). In contrast, the probiotic treated group on average had 7.1 viable offspring (standard deviation of 1.10), and 1.5 resorbed fetuses (standard deviation of 1.18).

TABLE 2
Probiotic Composition Resorbed Fetus in Mice

| Control Treated with Saline | | | Probiotic Treated | | |
|---|---|---|---|---|---|
| Mouse # | viable | resorbed | Mouse # | viable | resorbed |
| 1 | 4 | 3 | 1 | 7 | 2 |
| 2 | 2 | 5 | 2 | 8 | 0 |
| 3 | 3 | 5 | 3 | 6 | 2 |
| 4 | 4 | 6 | 4 | 7 | 2 |
| 5 | 3 | 7 | 5 | 8 | 1 |
| 6 | 2 | 4 | 6 | 7 | 0 |
| 7 | 3 | 3 | 7 | 9 | 2 |
| 8 | 3 | 3 | 8 | 7 | 3 |
| 9 | 4 | 5 | 9 | 5 | 3 |
| 10 | 2 | 8 | 10 | 7 | 0 |

Although the instant disclosure sufficiently describes inventive aspects and embodiments, the numerous references cited herein may be of assistance in understanding the background and the state of the art. Accordingly, all of the publications cited herein are hereby incorporated by reference into the present disclosure as if set forth in full herein.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for reducing a pregnancy complication or a risk of a pregnancy complication in a subject, the pregnancy complication selected from the group consisting of recurrent spontaneous abortions (RSA), preterm birth, low birth weight, preeclampsia, hemolysis elevated liver enzymes low platelets (HELP), premature rupture of the membrane, antepartum hemorrhage, placental abruption, chorioamnionitis, intrauterine growth restriction, placenta previa, sequelae of intraamniotic infection, and cerebral palsy, the method comprising:
administering to the subject a probiotic composition comprising Lactobacillus, Bifidobacterium, Escherichia, Saccharomyces, Streptococcus, Bacillus, or combinations thereof.

2. The method of claim 1, wherein the pregnancy complication is RSA.

3. The method of claim 2, wherein the RSA includes one or more symptoms selected from the group consisting of having one or more miscarriages in the first trimester of pregnancy, having a higher natural killer cell activity compared to an age-matched group of women with one or more successful pregnancies, having a deficient T regulatory cell activity compared to an age-matched group of women with one or more successful pregnancies, having a higher number of circulating natural killer cells as compared to a group of age-matched women with one or more successful pregnancies, and having a lower number of circulating T regulatory cells as compared to a group of age-matched women with one or more successful pregnancies.

4. The method of claim 3, wherein said natural killer cell activity comprises an ability to induce death in vitro in a cell type susceptible to natural killer cell mediated killing.

5. The method of claim 3, wherein said T regulatory cell activity is quantified by an ability to inhibit a mixed lymphocyte reaction, an ability to inhibit proliferation of a lymphocyte after stimulation or an ability to inhibit cytokine production of a lymphocyte after stimulation.

6. The method of claim 5, wherein said cytokine is selected from the group consisting of Interferon gamma, TNF-α, IL-12, IL-15, IL-17, IL-2, and IL-21.

7. The method of claim 5, wherein said T regulatory cell possess ability to stimulate production of an anti-inflammatory cytokine selected from the group consisting of IL-4, IL-10, IL-13, IL-20, and TGF-β.

8. The method of claim 3, wherein said natural killer cells express a marker selected from the group consisting of CD16, CD56, perforin, and CD94.

9. The method of claim 3, wherein said T regulatory cells express a marker selected from the group consisting of FoxP3, TGF-β, LAG, and CD73.

10. The method of claim 2 wherein said preterm birth is birth before 37 weeks of gestation.

11. The method of claim 2, wherein said risk of preterm birth comprises an increased vaginal or systemic concentration of one or more compound selected from the group consisting of sialidase, prolidase, glycosyltransferase types I, II and IV, monocyte chemotactic protein-1, matrix metalloproteases I, VIII and IX, IP-10, IL-6, IL-1 beta, TNF-alpha, fetal fibronectin; thrombin-antithrombin complex, and salivary estriol, as compared to a group of age-matched women having one or more successful pregnancies.

12. The method of claim 2, wherein said risk of preterm birth comprises a decreased vaginal or systemic concentration of one or more compound selected from the group consisting of maternal serum placental leucine amniopeptidase (P-LAP), IL-10, insulin-like growth factor-binding protein-1 (IGBP-1), Pregnancy associated plasma protein-A (PAPPA), and Corticotropin-releasing hormone (CRH), as compared to a group of age-matched women having one or more successful pregnancies.

13. The method of claim 1, wherein said probiotic composition comprises a lactic acid bacteria.

14. The method of claim 13, wherein said lactic acid bacteria is selected from the group consisting of Lactobacillus, Leuconostoc, Pediococcus, Lactococcus, Aerococcus, Carnobactehum, Enterococcus, Oenococcus, Teragenococcus, Vagococcus, and Weisella.

15. The method of claim 1, wherein said probiotic composition comprises one or more bacteria selected from the group consisting of Streptococcus thermophiles, Lactobacillus reuteri, Bifidobacterium bifidium, Lactobacillus acidophilus, and Lactobacillus casei.

16. The method of claim 1, wherein said probiotic composition comprises Streptococcus thermophiles, Lactobacillus reuteri, Bifidobacterium bifidium, Lactobacillus acidophilus, and Lactobacillus casei.

17. The method of claim 1, wherein said probiotic combination is about 10⁶ to about 10¹² colony forming units (CFU) bacteria/gram.

18. The method of claim 1, wherein said probiotic combination is administered orally.

19. The method of claim 18, wherein said probiotic combination is administered together with a food supplement.

20. The method of claim 1, wherein dosage and frequency of administration is based on immune response of the subject, and wherein said immune response is measured using immune parameters selected from the group consisting of T cell proliferative response to a mitogen, T cell cytokine production in response to a mitogen, NK cytotoxicity, level of complement fixing antibodies, B cell production of complement fixing antibodies, and production of inflammatory cytokines.
